(19) United States
(12) Patent Application Publication
(10) Pub. No.: US 2017/0119977 A1
(43) Pub. Date: May 4, 2017

(54) VALVED CATHETERS AND RELATED METHODS

(71) Applicant: B. Braun Melsungen AG, Melsungen (DE)

(72) Inventor: Hui Kuun TEOH, Penang (MY)

(73) Assignee: B. Braun Melsungen AG, Melsungen (DE)

(21) Appl. No.: 14/928,758

(22) Filed: Oct. 30, 2015

(51) Int. Cl. A61M 5/38; A61M 39/10; A61M 39/24; A61M 25/00; A61M 25/06

(52) U.S. Cl. CPC A61M 5/38; A61M 25/0097; A61M 25/0631; A61M 39/24; A61M 25/0014; A61M 39/10; A61M 25/0693; A61M 2039/1038

(57) ABSTRACT

Catheter devices are described. The catheter devices can have a catheter hub, a needle hub, and a bypass system for controlling fluid flow around a barrier. The bypass system can include flow ports for routing flow around the barrier. A filter can be placed in the path fluid flow in the distal direction to filter out contaminants or other unwanted materials prior to discharging out the catheter tube. A gas permeable membrane can be included to expel gas from the catheter device. A needle guard is usable with the needle device to block a tip of the needle.

### FIELD OF ART

The disclosed invention generally relates to intravenous (IV) infusion devices, including IV and arterial catheters. In particular, catheter devices and related methods having a filter system are disclosed.

### BACKGROUND

Needle devices are commonly used for a variety of infusion therapies, including infusing fluids into a patient, withdrawing blood from a patient, or monitoring various parameters of the patient's vascular system, to name a few examples. A catheter tube connected to a catheter hub is typically connected to a male Luer connector that connects to an IV tubing. Blood control catheters include an internal blood control regulator, such as a valve, that is opened by the insertion of a male Luer or other inserted into a proximal end of the catheter hub. Thus, following placement of the catheter tube into the vasculature of a patient, an IV fluid source can be connected to the catheter hub, opening the blood control valve. Once connected, fluid from the IV source can begin flow into a patient through the catheter. Conventionally speaking, the proximal end is the end closer to the practitioner and the distal end is further away from the practitioner.

For neonates, ICU patients, and patients highly susceptible to fungi, spores, bacteria, particles, or other harmful elements, an IV filtering system can be used to protect the patient during the application of drugs and infusions. Air embolisms can lead to complications.

### SUMMARY

Aspects of the present disclosure include catheter devices. The catheter devices described herein can be a catheter assembly. The catheter assembly can be an over the needle catheter or an IV catheter assembly. The catheter assembly can include a catheter hub comprising a catheter hub body, a catheter tube attached to a distal end of the catheter hub body, a barrier located in an interior cavity of the catheter hub body, and a needle projecting through the barrier, through the catheter tube, and having a needle tip at a distal end of the needle extending distally of a distal end of the catheter tube in a ready to use position. The needle has a proximal end attached to a needle hub. The barrier can allow proximal flow through the barrier for aspiration, and prevent distal flow through the barrier, thereby directing the distal flow from the proximal end of the catheter hub through a filter to the catheter tube to the patient.

The catheter device can further comprise a gas permeable membrane communicating with the catheter hub for expelling air outside the catheter hub.

The barrier can separate the interior cavity of the catheter hub body into a first interior cavity and a second interior cavity.

The barrier can have one or more extensions or one or more flaps separated from one another by one or more slits.

The bypass can comprise a first component and a second component and wherein the first component can have a flap. The flap of the first component can cover a port on the first component and/or cover the second port of the catheter hub.

The catheter hub can further have an enclosed compartment extending laterally from the catheter hub body. The enclosed compartment can communicate with the interior cavity of the catheter hub body.

The filter can be located in the enclosed compartment for filtering contaminants from entering the catheter tube. The first interior cavity can communicate with the filter through an inlet passage, and the filter can communicate with the second interior cavity through an outlet passage.

The filter can separate the enclosed compartment into a first compartment and a second compartment. The first interior cavity can communicate with the first compartment through an inlet passage and the second compartment can communicate with the second interior cavity through an outlet passage.

The filter can be clamped between the first compartment and the second compartment.

The second compartment and the catheter hub can be molded as a single component, with the first compartment attached over the filter and second compartment to form the enclosed compartment.

The catheter device can further comprise at least one gas permeable membrane attached to the catheter hub for expelling air outside the catheter hub.

The at least one gas permeable membrane can face a direction having at least a vertical direction component opposite earth or the ground.

The at least one gas permeable membrane can be made from a PTFE material.

The at least one membrane can cover the at least one gas permeable membrane to prevent touch contamination of the gas permeable membrane.

The at least one membrane can be located on the enclosed compartment and the membrane cover can be molded together with the enclosed compartment.

The at least one gas permeable membrane can be ultrasonically welded to the side of the enclosed compartment opposite the membrane cover.

The distal flow to the patient can be infusion fluid under the force of gravity pressure.

The needle can project through a slit through a center of the barrier.

The barrier can be made of an elastomeric or elastic material.

The barrier can be a check valve, such as a duckbill valve, a Heimlich valve, or a joker valve.

The catheter device can further comprise an additional enclosed compartment, such as a second enclosed compartment, having an additional filter located therein extending laterally from the catheter hub body.

The additional enclosed compartment can extend laterally from the catheter hub body opposite the other enclosed compartment.

The catheter device can further comprise a needle guard comprising a proximal wall, two arms, and two distal walls configured for covering the needle tip in a secured position.

The needle guard can be disposed in the interior cavity of the catheter hub body in the ready to use position.

The needle can have a change in profile located proximally of the needle tip. The catheter hub can have a change in profile located in the interior cavity thereof.

A blood stopper can be attached to a proximal end of the needle hub to prevent blood exposure during flashback.

Another aspect of the present disclosure includes a catheter device which comprises a catheter hub comprising a catheter hub body, an enclosed compartment extending laterally from the catheter hub body, with the catheter hub body having a proximal end, an interior cavity, a distal end communicating with the proximal end through the interior cavity, and an inlet passage and an outlet passage communicating with the interior cavity of the catheter hub body with the enclosed compartment. The catheter device can further include a catheter tube attached to the distal end of the catheter hub, a gas permeable membrane attached to the catheter hub and configured for expelling air outside the catheter hub, a barrier blocking flow distally through the barrier, and separating the interior cavity of the catheter hub body into a proximal interior cavity and a distal interior cavity, a filter located in the enclosed compartment and configured for filtering contaminants from entering the distal interior cavity from the proximal interior cavity, and a needle projecting through the barrier, through the catheter tube. The needle has a needle tip extending distally of a distal end of the catheter tube in a ready to use position, and a proximal end attached to a needle hub.

The barrier can block flow distally through the barrier to force infusion flow to the patient from the proximal end of the catheter hub body through the proximal interior cavity, the inlet passage, the filter, the outlet passage, the distal interior cavity, and the catheter tube.

The infusion flow to the patient can be infusion fluid under gravitational force.

The barrier can allow proximal flow through the barrier to enable aspiration.

The needle can project through a slit through a center of the barrier.

The barrier can be made of an elastomeric or elastic material.

The barrier can be a check valve, such as a duckbill valve, a Heimlich valve, or a joker valve.

The gas permeable membrane can be facing a direction having at least a vertical direction component opposite earth or the ground.

The gas permeable membrane can be attached to the enclosed compartment.

The gas permeable membrane can be made of a PTFE material.

The catheter device can further comprise a membrane cover on a side of the enclosed compartment covering the gas permeable membrane to prevent touch contamination of the gas permeable membrane.

The membrane cover can be molded together with the enclosed compartment.

The gas permeable membrane can be ultrasonically welded to the side of the enclosed compartment opposite the membrane cover.

The filter can separate the enclosed compartment into a first compartment communicating with the proximal interior cavity and a second compartment communicating with the distal interior cavity.

The filter can be clamped between the first compartment and the second compartment.

The second compartment and the catheter hub can be molded as a single component. The first compartment can be attached over the filter and the second compartment to form the enclosed compartment.

The catheter device can further comprise an additional enclosed compartment extending laterally from the catheter hub body. The additional enclosed compartment can have an additional filter located therein.

The additional enclosed compartment can extend laterally from the catheter hub body opposite the other enclosed compartment.

The catheter device can further comprise a needle guard comprising a proximal wall, two arms, and two distal walls configured for covering the needle tip in a secured position.

The needle guard can be disposed in the interior cavity of the catheter hub body in the ready to use position.

The needle can comprise a change in profile located proximally of the needle tip.

A blood stopper can be attached to a proximal end of the needle hub to prevent blood exposure during flashback.

Another aspect of the present disclosure includes a method for manufacturing the catheter device. The method can comprise attaching a catheter tube attached to a distal end of the catheter hub, said catheter hub comprising a hub body comprising an interior cavity and a proximal end communicating with the distal end through the interior cavity, placing a barrier in the interior cavity of the catheter hub, said barrier allowing flow proximally through the barrier, but preventing flow distally through the barrier and redirecting the flow distally through a filter, and projecting a needle through the barrier and through the catheter tube so that a needle tip of the needle extends distally of a distal end of the catheter tube in a ready to use position; said needle having a proximal end attached to a needle hub.

Yet another aspect of the present disclosure includes a method of using a catheter device comprising a catheter hub with a catheter tube, a needle attached to a needle hub, and a barrier located in an interior cavity of the catheter hub. The method can comprise removing the needle and needle hub from the catheter hub, barrier, and catheter tube, placing a male medical implement into a proximal opening of the needle hub, and infusing fluid from the male medical implement through a filter prior to the patient or withdrawing fluid from the patient to the male medical implement. The barrier can prevent fluid flowing distally through the barrier in the infusing step, and allow fluid to flow proximally through the barrier from the patient to the male medical implement.

A still further aspect of the present disclosure is a catheter device comprising: a catheter hub comprising a catheter hub body, the catheter hub body having a proximal end, an interior cavity, and a distal end communicating with the proximal end; a catheter tube attached to the distal end of the catheter hub body; a barrier located in the interior cavity of the catheter hub body and separating the interior cavity into a distal interior cavity and a proximal interior cavity, the barrier allowing flow in a proximal direction for aspiration but preventing flow in a distal direction through the barrier, a bypass comprising a first port for directing flow outside the interior cavity and around the barrier and a second port spaced from the first port for direction flow back into the interior cavity; and a needle projecting through the barrier, through the catheter tube, and having a needle tip extending distally of a distal end of the catheter tube in a ready to use position, the needle having a proximal end attached to a needle hub.

The catheter device can further comprise a gas permeable membrane communicating with the catheter hub and configured for expelling air outside the catheter hub.

The catheter device wherein the first port and the second port can be in fluid communication with an enclosed compartment.

The catheter device wherein the enclosed compartment can extend laterally from the catheter hub body.

The catheter device can further comprise a filter located in the enclosed compartment, the filter can filter contaminants passing through the enclosed compartment.

The catheter device wherein the first port and the second port can be located on different elevations relative to a lengthwise axis of the catheter hub.

The catheter device wherein the filter can separate the enclosed compartment into a first compartment and a second compartment.

The catheter device can further comprise at least one gas permeable membrane attached to the enclosed compartment for expelling air.

The catheter device wherein the at least one gas permeable membrane can be made from PTFE.

The catheter device can further comprise a membrane cover covering the at least one gas permeable membrane to prevent touch contamination of the at least one gas permeable membrane.

The catheter device can further comprise a needle guard slidably mounted relative to the needle for covering the needle tip.

The catheter device wherein the needle guard can comprise a proximal wall with an opening and at least one resilient arm.

The catheter device wherein the barrier can comprise a duckbill valve, a Heimlich valve, or a joker valve.

The catheter device can further comprise an interior change in profile in the interior cavity of the catheter hub for engaging a needle guard.

The catheter device wherein the bypass can comprise a first component attached to a second component comprising a compartment.

The catheter device wherein the second component can comprise a bottom wall and a sidewall.

Projections can be provided in the interior cavity of the catheter hub to retain the barrier. The projections can embody two continuous projections that are spaced from one another by a gap or can comprise a plurality of spaced apart bumps placed circumferentially around the interior cavity of the catheter hub body for receiving a base of the barrier. In an alternative embodiment, the barrier has external projections for engaging two corresponding spaced apart grooves formed on or in the catheter hub.

A still further aspect of the present disclosure is a method of manufacturing a catheter device comprising: attaching a catheter tube to a distal end of a catheter hub, said catheter hub comprising a catheter hub body comprising an interior cavity, a proximal end and a distal end; placing a barrier in the interior cavity of the catheter hub to define a proximal interior cavity and a distal interior cavity, said barrier is sized and shaped to allow fluid to flow proximally through the barrier from the distal interior cavity to the proximal interior cavity but prevents fluid from flowing distally through the barrier; projecting a needle through the barrier and through the catheter tube so that a needle tip of the needle extends distally of a distal end of the catheter tube in a ready to use position; said needle having a proximal end attached to a needle hub; and providing a bypass around the barrier for fluid flow around the barrier.

The method can further comprise placing a filter in a chamber of the bypass.

The method can further comprise placing a needle guard slidably relative to the needle for covering the needle tip in a protective position.

The method wherein the bypass can comprise a first port in communication with the proximal interior cavity, a second port in communication with the distal interior cavity, and an enclosed compartment in communication with both the first port and the second port.

Yet another aspect of the present disclosure is a method of using a catheter device comprising a catheter hub with a catheter tube, a needle attached to a needle hub, and a barrier located in an interior cavity of the catheter hub, said method comprising: removing the needle and needle hub from the catheter hub, the barrier, and the catheter tube; placing a male medical implement into a proximal opening of the catheter hub; and passing fluid from the male medical implement through a filter prior to discharging the fluid out through the catheter tube; and wherein the barrier prevents fluid from flowing distally through the barrier but allows fluid to flow proximally through the barrier.

### BRIEF DESCRIPTION OF THE DRAWINGS

These and other features and advantages of the present devices, systems, and methods will become appreciated as the same becomes better understood with reference to the specification, claims and appended drawings wherein:

FIG. 1 is an assembled perspective view of an embodiment of a catheter device of the present disclosure;

FIG. 2 is an exploded perspective view of the catheter device of FIG. 1;

FIG. 3 is a cross-sectional side view of the catheter device of FIG. 1 without the needle, the needle guard, and the needle hub.

FIG. 4 is similar to FIG. 3 but shown from a different perspective;

FIG. 5 is an embodiment of a barrier for use with the catheter device of FIG. 1;

FIG. 6 is a cross-sectional side view of the catheter device of FIG. 1 in a ready to use position;

FIG. 7 is a cross-sectional side view of the catheter device in a secured position; and

FIG. 8 is a partial cross-sectional side view of the catheter device similar to FIG. 7 shown with a flow path depicting a direction of fluid flow during infusion.

FIG. 9 is a perspective view of an alternative barrier having a plurality of flaps.

FIG. 10 is a perspective view of yet another alternative barrier having at least one extension or flap.

FIG. 11 is a cross-sectional side view of the catheter device of FIG. 1 without the needle, the needle guard, and the needle hub and with the barrier of FIG. 10.

FIG. 12 is a cross-sectional side view of the catheter device of FIG. 1 without the needle, the needle guard, and the needle hub and with the barrier of FIG. 9.

FIG. 13 is an exploded perspective view of the catheter device of FIG. 1 with an alternative cover and barrier.

### DETAILED DESCRIPTION

The detailed description set forth below in connection with the appended drawings is intended as a description of the presently preferred embodiments of catheter devices provided in accordance with aspects of the present assemblies, systems, and methods and is not intended to represent the only forms in which the present devices, systems, and methods may be constructed or utilized. The description sets forth the features and the steps for constructing and using the embodiments of the present assemblies, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

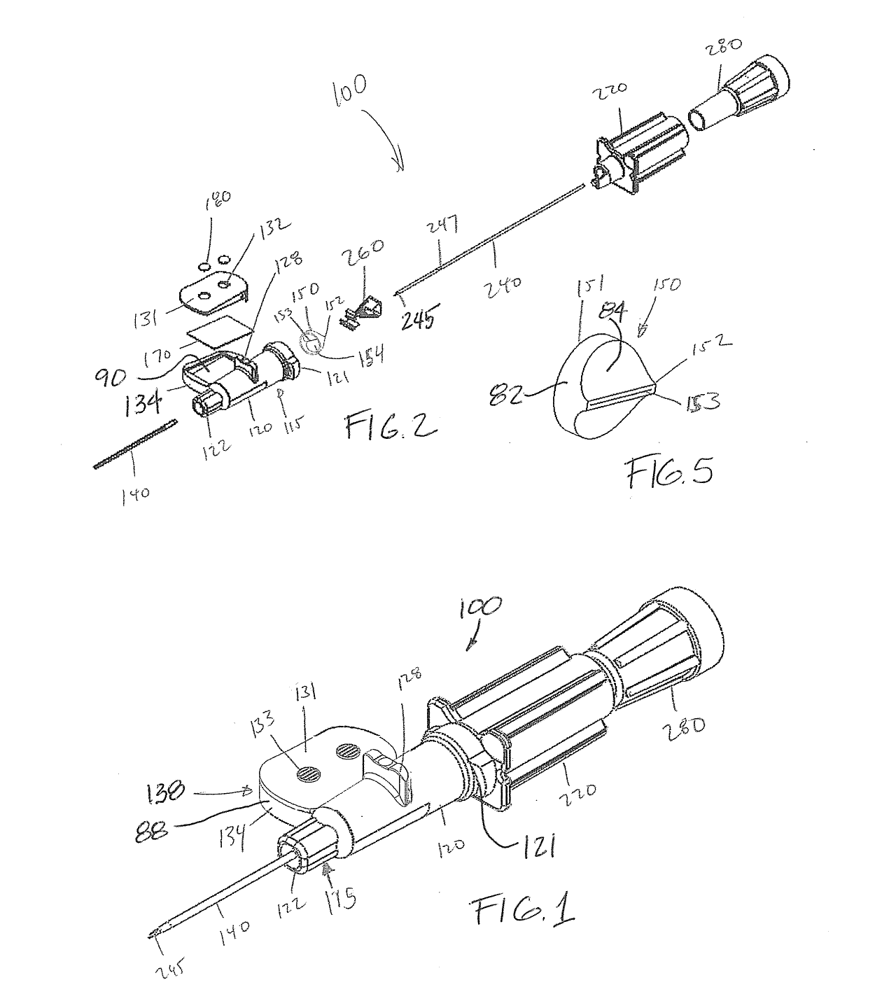

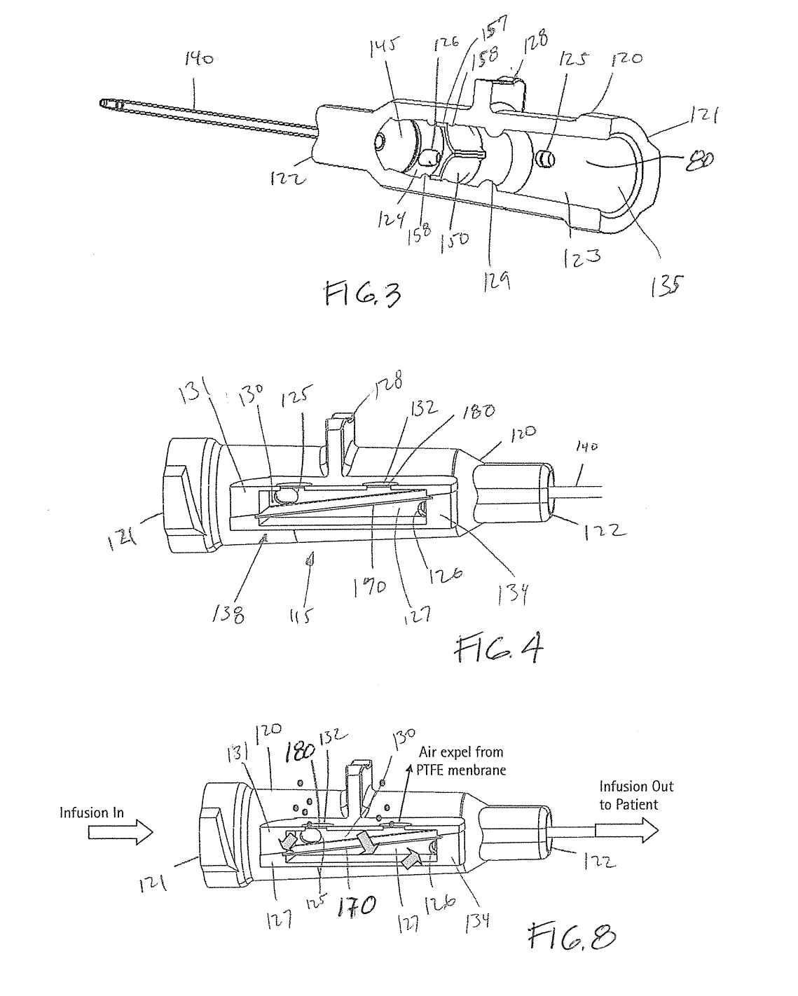

With reference now to FIGS. 1 and 2, a catheter device 100, such as an over-the-needle catheter assembly or an IV catheter assembly, provided in accordance with aspects of the present disclosure are shown. FIG. 1 shows the catheter device 100 in an installed or ready to use position while FIG. 2 shows an exploded view of the catheter device.

In an example, the catheter device 100 comprises a catheter hub 115 having a catheter hub body 120, a catheter tube 140 extending distally from a distal end 122 of the catheter hub body 120, a barrier 150 located in an interior cavity or bore (FIG. 3), and a needle 240 extending from a needle hub 220 and projecting through the proximal end 121 of the catheter hub body 120, the barrier 150, and the catheter tube 140 in the ready to use position of FIG. 1.

As shown in FIG. 3, when the barrier 150 is positioned inside the bore or interior cavity 80, it separates the interior cavity 80 into a proximal interior cavity 123 and a distal interior cavity 124, as further discussed below.

A proximal end of the catheter tube 140 can attach the catheter hub 120 using a bushing 145 (FIG. 3) or other conventional means. The needle 240 has a needle tip 245 extending out a distal end of the catheter tube 140 in the ready to use position, and a proximal end attached to the needle hub 220. The needle 240 projects distally from a nose section of the needle hub 220 and in through the catheter hub body 120 and the catheter tube 140 with the needle tip 245 extending out the distal end of the catheter tube for penetrating the epidermal layer of a patient and accessing the vasculature of the patient.

The needle hub 220 has a flashback chamber and a proximal opening, which can be closed by a vent plug or a blood stopper 280 attached at the proximal opening of the needle hub 220. Optionally, a needle guard 260 is provided.

As shown, the needle guard 260, if incorporated, is positioned inside the interior cavity of the catheter hub body 120. The needle guard 260 may be similar to the needle guard disclosed in U.S. Pat. No. 6,616,630, which has two arms each with a distal wall for blocking the needle tip and a proximal wall comprising a perimeter defining a proximal opening having the needle 240 passing therethrough. The needle guard 260 is configured for covering the needle tip 245 in a secured position or protective position (FIG. 7), such as when the needle is removed from the catheter hub and the needle guard protects the needle tip from unintended needle sticks.

The needle 240 can have a change in profile 247, such as a crimp, a bulge, a sleeve, or a material buildup, for engaging the perimeter with the proximal opening on the proximal wall of the needle guard following placement of the catheter tube 140 into a patient's vein. In some examples, the needle guard 260 is omitted from the catheter device 100. When incorporated, the needle guard 260 can be positioned completely inside the catheter hub body 120, partially inside the catheter hub body 120, or completely outside the catheter hub body 120, such as in a shroud or a separate hub housing between the catheter hub body 120 and the needle hub 220.

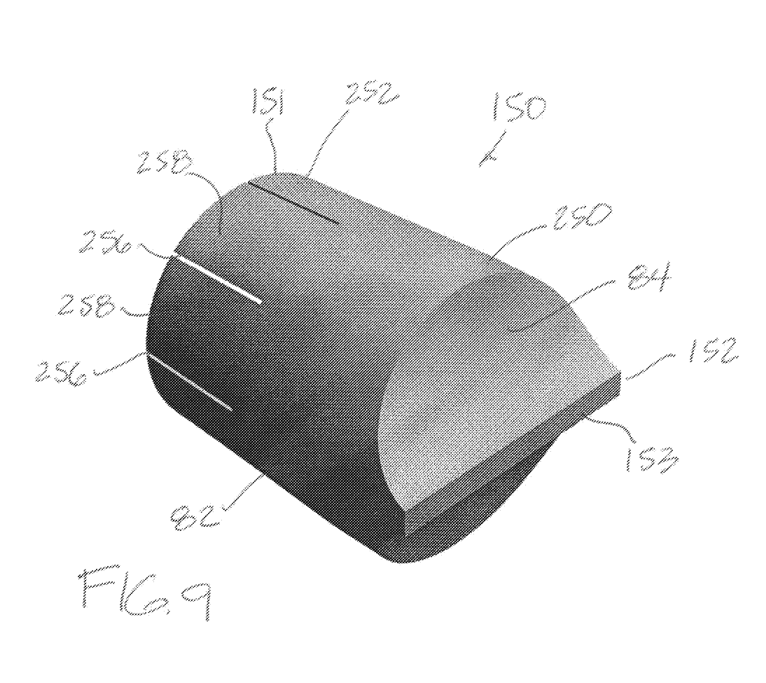

A change in profile 129 is provided in the bore of the catheter hub 115, which can include a first interior diameter located next to a larger second interior diameter, for retaining the needle guard 260 in the ready to use position (as shown in FIGS. 2 and 6) and for retaining the needle guard 260 during retraction of the needle 240 following successful venipuncture to a secured position. The change in profile 129 can also be viewed as an inward projection located adjacent a recess inside the bore of the catheter hub.

The proximal end 121 of the catheter hub 120 can have a female Luer with external threads, also known as a threaded female Luer. The female Luer connector is thus configured to matingly receive a male Luer connector, such as an IV line, a Luer access connector, a syringe tip, a vent plug, an IV set, an extension set, another known connector, or future-developed IV devices. Each of these components can be sized and configured in conformity with at least some of the International Standards Organization (ISO) standards for female and male Luer connections under current or future standards. For discussion purposes, any one of these components or the class of these components can be referred to as a male medical implement or a male connector.

A tab 128 extending from the catheter hub body 112 can be used as leverage when handling the device 100, such as to push against during insertion or removal of the needle 240. Optionally, the tab 128 can be omitted.

Referring now to FIG. 3, the barrier 150 is shown located in the interior cavity 80 of the catheter hub body 120. In one example, the barrier 150 is pressed against the interior wall surfaces 157 of the interior cavity 80 thereby forming a seal between the perimeter of the barrier 150 and the interior wall surfaces 157.

The barrier 150 can be secured between two grooves or two projections 158 formed interiorly of the hub body 120 and distal of the interior change in profile 129. The two grooves or two projections 158 in the interior defines a gap for receiving the barrier 150 and retain the barrier therein against axial displacement. For example, the width of the barrier 150 can be located between the gap defined by the two projections 158.

The barrier 150 can be located within the interior cavity of the catheter hub 120 distal of the female Luer taper so as to not interference with a male medical implement inserted into the proximal opening 135 at the proximal end 121 of the catheter hub body 120 following successful venipuncture. The projections 158 can embody two continuous projections that are spaced from one another by a gap or can comprise a plurality of spaced apart bumps placed circumferentially around the interior cavity of the catheter hub body 120. In an alternative, the barrier 150 has external projections for engaging two corresponding spaced apart grooves 158 formed on or in the catheter hub.

In an example, the barrier 150 can be made of an elastomeric material, such as from a silicone material, and is biocompatible and elastic so that the barrier 150 can be slid between the two circumferential projections 158 when assembling the barrier 150 into the catheter hub 115. In some examples, the projections 158, or one projection, can be omitted and the barrier can be positioned in the interior cavity 80 via interference.

The barrier 150 can separate the interior cavity 80 of the catheter hub body 120 into a proximal interior cavity 123 and a distal interior cavity 124. The barrier 150 can be similar to a one-way valve or check valve that allows proximal flow through the barrier 150 for aspiration but prevents distal flow, such as distal infusion fluid flow, through the barrier 150. Flow can seep through the barrier in small quantities but can expect to behave as described. In an example, the barrier 150 is a duckbill valve, a Heimlich valve, or a joker valve.

The barrier 150 can block fluid flow through the catheter hub 115 in the distal direction. A flow bypass system or simply bypass can be provided with the catheter hub body 120 to permit infusion or other fluid flow from the proximal end 121 of the hub body 120 to the distal end 122 of the hub body 120 and out through the catheter tube 140 to provide fluid flow around the barrier 150. In an example, a bypass is provided around the barrier 150. The bypass can comprise two passages. The bypass can be external of the catheter hub 115. For example, the barrier 150 can cause distally directed flow to flow laterally out the catheter hub 115 so as to bypass the barrier 150. In some examples, as the flow is directed outside the catheter hub body 120, the flow can pass through a filter 170 before exiting the catheter tube 140 and out to the patient.

In an example, the filter 170 can be made from a polyether-sulfone membrane. The filter 170 can be made from a positively charged membrane capable of absorptive separation. Endotoxins with their negative surface charge can be retained from entering to the patient when passed through the positively charged membrane, although the endotoxin size can be smaller than the filter pore size. Screen pore size can be selected to about 17 to about 260 micron (μm). In some examples, microaggregate filters can be used having pore size of about 20-40 micron. The filter 170 can be configured for filtering out contaminants, such as fungi, spores, bacteria, and particles. The filter 170 can also be selected to reduce air embolism. Thus, for example, the barrier 150 can force the infusion fluid to pass through the filter 170 before entering the patient's vasculature. In an example, a filter size or pore size indicator can be provided on the needle device 100. For example, a sticker can be placed on the exterior of the first component 131 to indicate the size of the filter or the pore size located inside the enclosed compartment 138.

The filter 170 can be located so as not obstruct the needle 240 when the needle projects through the catheter hub and out the catheter tube in the ready to use position, as shown in FIG. 1. In a particular example, the filter 170 is located in a compartment separate from the interior cavity 80 of the catheter hub body 120, as further discussed below. Alternatively, the filter can be located in the interior cavity 80 of the hub body 120 and the bypass is located externally of the hub body 120.

FIG. 5 illustrates one embodiment of the barrier 150 having a body 82 with a cylindrical base 151, an end wall 84 with a tip 152, and a slit 153 extending along the center portion of the tip 152 and through the end wall 84. The slit 153 is in communication with an interior cavity 154 (FIG. 2) of the barrier 150. In another example, instead of a single slit 153, the barrier 150 can have one or more holes or one or more slits 153 defined through the end wall 84 to enable the needle 240 to pass through the barrier 150. The taper angle of the tip 152 of the barrier and the material of the barrier 150 can be selected to minimize drag with the needle 240 when the needle retracts proximally away from the catheter hub 115, such as following successful venipuncture. In the illustrated embodiment, the tip 152 faces proximally towards the proximal opening 135 of the catheter hub body 120.

During aspiration, such as when a male medical implement is inserted into the proximal end 121 of the catheter hub body 120 and fluid is drawn proximally into the male medical implement, a pressure drop is experienced at the proximal interior cavity 123 caused by a suction effect, such as by retracting a plunger of a syringe proximally of the syringe barrel. This in turn allows higher fluid pressure distally of the barrier 150 to open the barrier 150, such as push through the slit 153, and fluid then flows into the lower pressure region proximally of the barrier 150. Because the pressure at the distal interior cavity 124 of the catheter hub 115, which is similar to the pressure at the interior cavity 154 of the barrier 150, is greater than the pressure at the proximal interior cavity 123 of the catheter hub 115, the difference in pressure will cause the slit 153 to open and for higher pressure region to flow to the lower pressure region. That is, the slit 153 will open to allow blood collection from the patient through the catheter tube 140, to the distal interior cavity 124, into the interior cavity 154 of the barrier 150, through the slit 153 of the barrier, and into the proximal interior cavity 123 of the catheter hub 115. Because fluid tends to flow along a path of least resistance, it will flow through the slit 153 during aspiration rather than through the side passages 125, 126 and the filter 170.

When no vacuum and no infusion are applied at the proximal opening 135 of the catheter hub 115, proximally directed flow can exit the distal interior cavity 126 through the second port or passage 126 and into the enclosed compartment 138. However, because the filter 170 located in the enclosed compartment 138 presents a relatively large resistance to flow, fluid flowing proximally from the distal interior cavity 124, if any, through the filter 170 to the proximal interior cavity 123 is minimal. In still other examples, a one-way flap can be incorporated at the second port or passage 126 at the distal interior cavity 124 to limit flow into the enclosed compartment 138. For example, the flap can flex towards the distal interior cavity 124 of the catheter hub to permit flow from the enclosed compartment 138 but is pushed against a lip, a flange, or a wall surface when fluid is pushed from the distal interior cavity 124 towards the enclosed compartment 138 to prevent or limit flow into the enclosed compartment. Aspects of the present disclosure involving a flap are further discussed below with reference to FIGS. 9-13.

During infusion when fluid pressure at the proximal interior cavity 123 of the catheter hub 115 is relatively higher than the distal interior cavity 124, the barrier 150 will close. Fluid pressure will cause surfaces at the extended tip 152 on the end wall 84 of the barrier 150 to press close and to close the slit 153. With the barrier 150 being closed by infusion fluid pressure, fluid will need to find a different path to flow from the proximal interior cavity 123 to the distal interior cavity 124. In the present application, the different path can be a bypass flow path. The bypass flow path can direct fluid flow from the proximal interior cavity of the catheter hub around the barrier and to the distal interior cavity of the catheter hub. The bypass flow path can direct flow outside of the catheter hub and then back into the catheter hub.

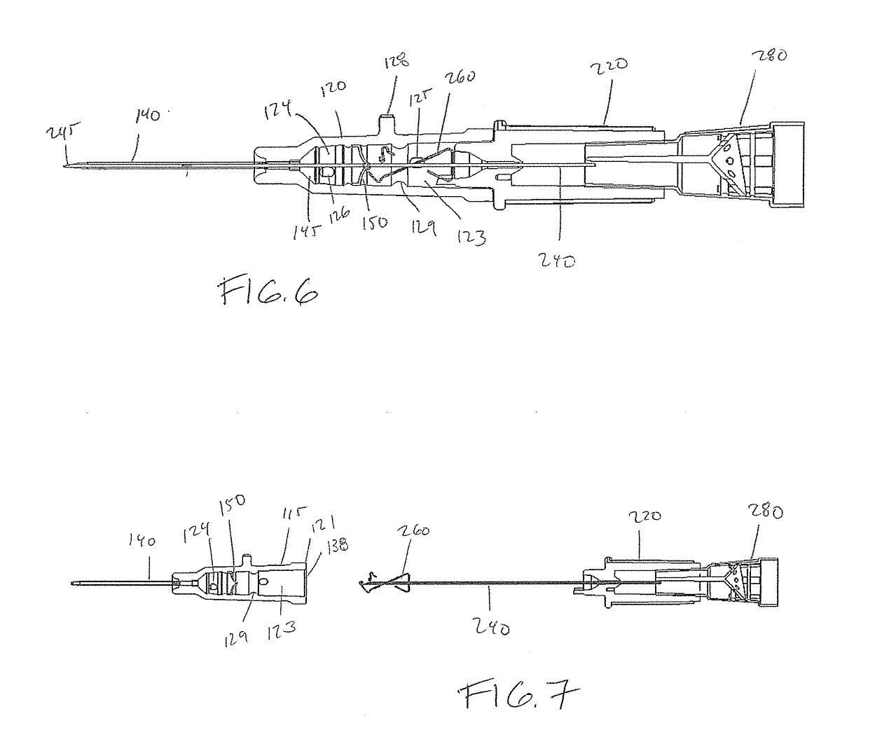

Referring now to FIG. 4 in addition to FIGS. 1 and 2, the catheter hub 115 can include a bypass for fluid flow to flow from the proximal interior cavity 123 to the distal interior cavity 124 when the barrier 150 is closed. In an example, the bypass can include an enclosed compartment 138 located laterally of the catheter hub body 120. For example, the catheter hub 115 can have an exterior surface and a lengthwise axis and the enclosed compartment 138 can attach to the exterior surface along the lengthwise axis. The enclosed compartment can 138 be unitarily formed with the catheter hub 115 or can be separately formed and subsequently attached to the catheter hub, such as by welding, bonding, using mechanical securement means, or combinations thereof.

The enclosed compartment 138 can communicate with both the proximal interior cavity 123 and the distal interior cavity 124 of the catheter hub body 120. For example, a passage can extend from the proximal interior cavity 123 of the catheter hub and the enclosed compartment 138 and another passage can extend from the distal interior cavity 124 of the catheter hub 115 and the enclosed compartment to establish fluid communication between the proximal interior cavity, the enclosed compartment, and the distal interior cavity.

The filter 170 can be located in the enclosed compartment 138 and filters fluid only after the fluid exits the proximal interior cavity 123 of the catheter hub. In an example, the passage that extends from the proximal interior cavity 123 of the catheter hub and the enclosed compartment 138 and another passage that extends from the distal interior cavity 124 of the catheter hub 115 and the enclosed compartment can comprise a first flow port or inlet passage 125 in communication with the proximal interior cavity 123 and a second flow port or outlet passage 126 in communication with the distal interior cavity 124.

The first or proximal interior cavity 123 can communicate with the filter 170 located in the enclosed compartment 138 through the first flow port or inlet passage 125, which directs fluid out the proximal interior cavity 123 of the catheter hub body 120 and into the enclosed compartment 138. Fluid then travels through the filter 170 and is filtered thereby.

The filter 170 can communicate with the second or distal interior cavity 124 through the second flow port or outlet passage 126, which directs fluid out from the enclosed compartment 138 into the distal interior cavity 124 of the catheter hub 115. The inlet passage 125 can allow the infusion fluid to flow from the proximal interior cavity 123 through the filter 170 and out the outlet passage 126 to the distal interior cavity 124 of the catheter hub and out the catheter tube and into the vasculature of the patient. Because the barrier 150 closes when it experiences relatively higher pressure on the proximal side of the end wall 84, infusion fluid is prevented from flowing through the slit 153 of the barrier in a distal direction when the barrier closes and is forced to flow through the inlet passage 125 and into the enclosed compartment 138, through the filter 170 located in the enclosed compartment 138, and out the outlet passage 126 and back into the catheter hub body 120, into the distal interior cavity 124 of the catheter hub body 120. From there, fluid can flow out through the catheter tube. Infusion fluid flow from a male medical implement or male connector connected at the proximal end of the catheter hub therefore bypasses the barrier 150, such as flow around the barrier 150, when directing flow from the proximal interior cavity 123 to the distal interior cavity 124 of the catheter hub 115.

In an alternative embodiment, the inlet passage 125 and the outlet passage 126 can communicate with one another through an external flow passage (now shown) that connects the two passages. For example, a tubing can have one end connected to the inlet passage 125 and another end to the outlet passage 126. As the tubing is too small to accommodate the filter 170, the catheter hub can be sized and shaped to accommodate the filter at the proximal interior cavity 123 for the embodiment with the external flow passage.

As shown with reference to FIGS. 2 and 4, the filter 170 can be positioned inside the chamber of the enclosed compartment 138 in such a way that it separates the chamber of the enclosed compartment 138 into a first or upper compartment or chamber 130 and a second or a lower compartment or chamber 127. As shown, the proximal interior cavity 123 of the catheter hub 115 can communicate with the upper compartment or upper chamber 130 through the inlet passage 125 while the distal interior cavity 124 of the catheter hub 115 can communicate with the lower chamber or compartment 127 through the outlet passage 126. The two passages 125, 126 can communicate with one another through the interior cavity of the enclosed compartment 138, which can have a filter 170 located therein. In an example, the filter 170 can have a planar surface and whereby the planar surface can be angled relative to the lengthwise axis of the catheter hub 115 when positioned inside the enclosed compartment 138. In an example, the filter 170 can have a proximal end located, elevation-wise, below the inlet passage 125 and a distal end located, elevation-wise, above the outlet passage 126. The filter 170 can have ends that rest on a ledge or a lever at each respective end of the interior of the enclosed compartment 138. One or both ends of the filter can be clamped to secure the filter inside the enclosed compartment. The ends of the filter 170 can be clamped by the first component 131 and a ledge or shoulder inside the second component 134.

The inlet passage 125 and the outlet passage 126 therefore can be located, elevation-wise, on different levels relative to a lengthwise axis of the catheter hub. In an example, the inlet passage 125 is located higher than the outlet passage 126, and both relative to the lengthwise axis of the catheter hub. In yet another example, the inlet passage 125 is located lower than the outlet passage 126, and both relative to the lengthwise axis of the catheter hub. The orientation of the filter 170 and the relative positions of the inlet passage 125 and the outlet passage 126, elevation-wise, define the upper chamber 130 and the lower chamber 127. The two passages can alternatively be located on the same level and the filter having a baffle or a bend to separate the two passages into two different chambers, such as a first chamber and a second chamber instead of an upper chamber and a lower chamber.

The enclosed compartment 138 can have a first component 131 and a second component 134. The second component 134 can be molded with the catheter hub body 120, such as being co-molded or singularly molded with the catheter hub body. The second component 134 can have a bottom wall 90 and a sidewall 88 surrounding the bottom wall 90 and defining a compartment for accommodating a filter. Part of the sidewall 88 can be a side of the catheter hub. Alternatively, the sidewall 88 can extend the entire circumference of the bottom wall and the upper edge of the sidewall 88 can have a lip for receiving the first component 131.

The first component 131 can be a plate or a flange that is sized and shaped to fit over and enclose the compartment of the second component 134. The first component 131 can snap into a rim and sealed against the lip along the upper edge of the sidewall 88 of the second component 134. The first component 131 can include features to clamp the filter 170 within the interior of the enclosed compartment. Detents, adhesive, ultrasonic welding or other securement means may be used to secure the first component 131 to the second component 134, after placing a filter inside the second component 134 to form the enclosed compartment 138.

The shape and size of the enclosed compartment 138 is not limited. In the illustrated embodiment, the enclosed compartment 138 can function as a wing for securing the catheter device to a patient following successful venipuncture. In another embodiment, two enclosed compartments 138 can be incorporated with the catheter hub, diametrically opposed about the lengthwise of the catheter hub. The two enclosed compartments 138 can additionally function as wings and can extend laterally from the catheter hub body 120 with each enclosed compartment having a filter 170 for filtering, as discussed above. For the embodiment with two compartments, inlet and outlet passages are understood to also be incorporated with the second enclosed compartment. Thus infusion fluid can bypass the barrier 150 and flow through two filters 170 instead of one filter 170. The size of the filter 170 or the size of each of the two filters can depend on many factors such as infusion fluid, desired flow rate, body weight of the patient, and/or the duration of filtration. A larger filter 170 can ensure a longer infusion usage and enhance the flow rate of the catheter device 100. In one example, a filter size of about 1.5 square centimeters per enclosed compartment 138 can have a usage time of up to about 96 hours for normal infusion. Thus, a filter size of 3 square centimeters, such as when incorporating two enclosed compartments 138 and two filters 170 with each at 1.5 square centimeters, can be achievable with two enclosed compartments 138 to increase usage time of well over 100 hours, such as over 120 hours or over 140 hours.

A gas permeable but liquid impermeable membrane 180 can be attached to the catheter hub body 120 to communicate with the interior cavity 80 thereof or attached to the enclosed compartment 138 and be in fluid communication with the interior chamber of the enclosed compartment 138, such as the upper chamber 130 of the enclosed compartment 138. The gas permeable membrane 180 is configured for expelling air, if any, from the interior of the catheter hub 115 and/or the enclosed compartment 138. The gas permeable membrane 180 can be made of any gas permeable material that allows only gas molecules to pass through but not liquid. In one example, the gas permeable membrane 180 can be made of polytetrafluoroethylene (PTFE) or polydimethylsiloxane (PDMS), to name a few examples.

The gas permeable membrane 180 should be attached at a position that permits gar or air to escape. One or more gas permeable membranes 180 can be used. In the illustrated embodiment, two circular gas permeable membranes 180 are attached, in a spaced apart relationship, in the holes 132 defined in the first component 131 to communicate with the upper compartment 130 of the enclosed compartment 138. A membrane cover 133 can cover each of the gas permeable membranes 180 to prevent touch contamination of the membranes 180, as shown in FIG. 1. The membrane covers 133 can each have any shape or design, such as ribs, crosses, or a plurality of angled louvers, aligned in a single direction to allow gases to pass through but prevent fingers from touching and contaminating the gas permeable membrane 180. The membrane cover 133 can be molded together with the upper compartment 130 of the enclosed compartment 138 or separately formed and subsequently attached thereto. The gas permeable membranes 180 can be ultrasonically welded to the side of the enclosed compartment 138 opposite the membrane cover 133 or be fitted thereto by interference fit. In some examples, the gas permeable membranes 180 can have different shapes, such as square or oval, and the number of gas permeable membranes 180 can be greater than two.

FIG. 6 shows a cut-away view of the catheter device 100 in the ready to use position, prior to removal of the needle hub 220, the needle 240, and the needle guard 260 from the catheter hub 115. The needle 240 extends through the slit 153 of the barrier 150. A tool could be used to pry open the slit 153 or used to squeeze the barrier 150 to open the slit 153 to allow the needle to pass therethrough during assembly of the catheter device in the ready to use position. For example, a special blade tool could be used to enter the slit 153 of the barrier 150 and then turned to open the slit 153 to allow the needle 240 to pass therethrough during assembly without damaging the barrier 150 or the needle tip 245.

In the ready to use position of FIGS. 1 and 6, the needle tip 245 can be used to insert the needle and the catheter tube into the patient to access the vasculature of the patient. Successful venipuncture occurs when the needle tip 245 and distal end of the catheter tube 140 enter the vasculature and blood flashback can be seen entering the lumen of the needle 240 and into the needle hub 220. Blood can be drawn into the blood stopper 280. The blood stopper 280 can be similar to the blood stopper disclosed in pending U.S. patent application Ser. No. 14/576,802, filed Dec. 19, 2014, the contents of which are expressly incorporated herein by reference. In an example, the blood stopper 280 can be used to draw in and subsequently dispense blood, such as to test or sample the blood. Secondary flashback can also be used to confirm successful venipuncture by moving the needle tip slightly in the proximal direction to allow blood to flow in the annular space between the needle and the catheter tube. Once successful venipuncture has occurred, the needle hub 220 with the needle 240 can be detached and removed from the catheter hub 115.

FIG. 7 shows the needle hub 220, the needle 240, and the needle guard 260 removed from the catheter hub 115, such as following successful venipuncture. After removal of the needle from the slit 153 of the barrier 150, the slit 153 will seal upon itself to prevent blood from flowing into the proximal interior cavity 123 of the catheter hub 115 or at least substantially limit blood from flowing into the proximal interior cavity. The shape of the tip 152 and the size of the slit 153 are such that normal blood pressure cannot force the barrier to open and therefore the proximal interior cavity 123 is sealed or substantially sealed from the distal interior cavity 124 from free flowing of fluid or blood.

The barrier 150 is configured to permit flow through the slit 153 in the proximal direction after a certain differential pressure across the barrier 150 is exceeded. For example, the barrier 150 can be designed such that when the needle hub 220 and needle 240 are removed from the catheter hub 115, the pressure of the blood pressing against the barrier 150 from the patient in the distal interior cavity 124 is greater than the pressure at the proximal interior cavity 123, but not great enough to cause the slit 153 to open and for fluid to flow through the barrier 150. Thus, fluid flow, such as blood flow, can be completely blocked or substantially blocked so that little or no fluid can pass through the slit 153 in the proximal direction when the barrier 150 is closed. However, when a male medical implement, such as a syringe, is inserted through the proximal opening 135 at the proximal end 121 of the catheter hub body 120, the vacuum caused by retracting the plunger on the syringe will decrease the pressure at the proximal interior cavity 123 to increase the pressure differential across the barrier 150. This in turn can cause the slit 153 of the barrier 150 to open and fluid to flow from the distal interior cavity 124, through the barrier 150, and into the proximal interior cavity 123.

Following successful venipuncture and as the needle 240 is retracted proximally from the catheter hub 115 and the needle 240 passes through the tip 152 of the barrier 150 through the slit 153, the barrier 150 can wipe the needle 240 thereby removing at least some of the blood or fluid from the outer surface of the needle 240. Thus, the barrier 150 can function as a wiper to wipe blood from the needle during retraction of the needle from the catheter hub 115. The needle 240 can be retracted proximally and removed from the catheter hub 115 to then allow a male medical implement to connect to the open proximal end 121 of the catheter hub body 120. During the needle removal, the change in profile on the needle 240 engages the proximal wall of the needle guard and retracts the needle guard with the needle. For example, the change in profile can engage a perimeter defining an opening on the proximal wall of the needle guard during the needle retraction. The needle guard covers the needle tip from unintended needle sticks. The male medical implement can be connected to an infusion fluid or source for delivering IV fluids to the patient. The male medical implement can also be a syringe to withdraw blood from the patient.

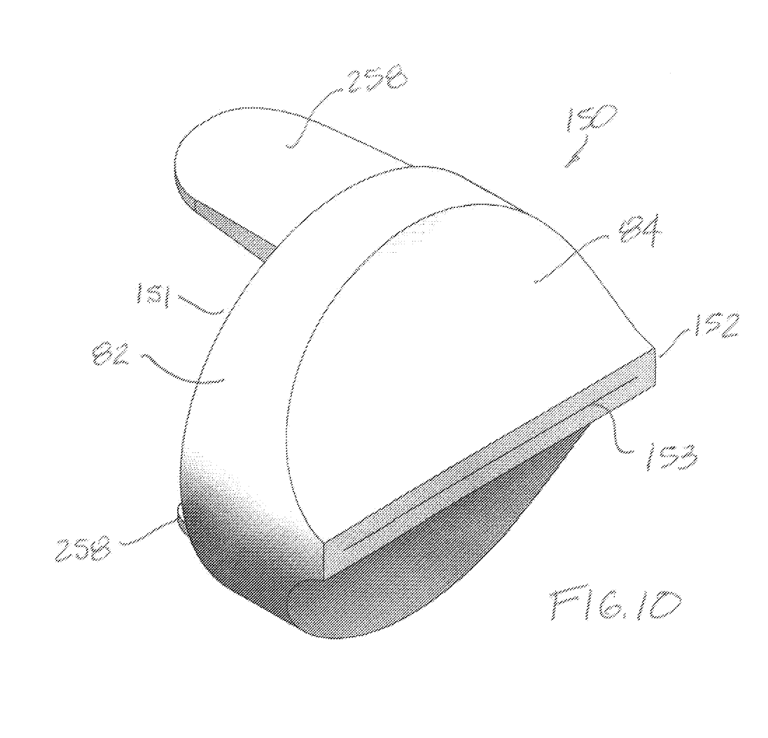

FIG. 8 shows the catheter device 100 after the needle 240 and the needle hub 220 have been retracted proximally from the catheter hub 115, such as following successful venipuncture. Infusion fluid can be introduced into the proximal end 121 of the catheter hub body 120, such as by inserting a male medical implement into the female Luer of the catheter hub and allowing infusion fluid to flow into the proximal interior cavity 123 of the catheter hub. Fluid pressure presses against the barrier 150 but due to the orientation of the barrier 150 and the shape of the tip 152, the pressure from the proximal interior cavity 123 forces the barrier 150 to close and the slit 153 to close on itself. The greater pressure at the proximal interior cavity 123 forces end wall 84 of the barrier 150 to shut and close the slit 153 to closed the barrier 150.

During infusion and because the barrier 150 blocks distal flow into the patient through the barrier 150 from the proximal interior cavity 123, infusion fluid flowing under gravity pressure flows from the proximal interior cavity 123 through the inlet passage 125, the upper compartment 130 of the enclosed compartment 138, the filter 170, the lower compartment 127, the outlet passage 126, the distal interior cavity 124, and then out the catheter tube 140 and into the patient. Thus, infusion fluid flow bypasses the barrier 150. As the infusion fluid flows through the upper compartment 130, air or gas can be expelled out through the one or more gas permeable membranes 180 and out through the openings or holes 132 of the membrane cover 133. If aspiration is desired, the infusion fluid line can be removed from the open proximal end of the catheter hub 115 and replaced with a medical device, such as a syringe, to draw blood from the patient. During aspiration, the blood from the patient flows directly from the distal interior cavity 124 through the barrier 150 and into the proximal interior cavity 123 as the pressure at the proximal interior cavity 123 is less than the pressure at the distal interior cavity 124 due to the vacuum generated by the syringe. Thus, the lower pressure causes sufficient differential pressure across the barrier to open the slit 153, as previously discussed.

With reference now to FIG. 9, a barrier 150 provided in accordance to alternative aspects of the prevent disclosure is shown. The barrier 150 has an end wall 84 with a tip 152 and a slit 153, similar to the barrier 150 shown and described with reference to FIG. 5. In the present embodiment, the body 82 has a cylindrical base 151 that has been extended or elongated compared to the cylindrical base 151 of FIG. 5. Furthermore, the cylindrical base 151 of the body 82 is tapered from a proximal end 250 to a distal end 252 such that the proximal end is larger in dimension than the distal end. This tapering in the distal direction allows the barrier 150 to be slid into the interior cavity 80 of a catheter hub 115 through the proximal opening 135 thereof during assembly. In other examples, the outside dimensions along the cylindrical base 151 are approximately constant or about the same but the wall thickness of the cylindrical base 151 gradually tapers so that the thickness at the distal end 252 is thinner than the thickness at the proximal end 250.

In the present embodiment, the cylindrical base 151 is elongated by an amount that is sufficient to cover the outlet port or second port 125 of the distal interior cavity 124 of the catheter hub when the barrier 150 is placed therein. The cylindrical base of the present barrier 150 embodiment can cover two or more second ports 125, such as in a medical device having two enclosed compartments, two filters, and two second ports. By covering the second port 125, blood flow into the distal interior cavity 124 of the catheter hub is isolated or prevented from flowing through the second port 125 to possibly occlude and/or contaminate the filter 170 located inside enclosed compartment 128. However, the body 82 can be provided with flaps 258 to permit infusion flow into the distal interior cavity 124, as further discussed below. The flaps are 258 flexible and can defect under fluid pressure.

In the example shown, the cylindrical base 151 is provided with a plurality of slits 256. The slits 256 can extend lengthwise relative to the length of the barrier 150. A flap 258 is formed between every two adjacent slits 256. In an example, the slits 256 can be evenly spaced around the circumference of the base 151 to form a plurality of flaps 258. In an example, eight spaced apart slits 256 are provided around the circumference of the base 151 to form eight flaps 258. However, different numbers of slits and flaps can be provided without deviating from the scope of the present disclosure, such as ten slits and ten flaps or twelve slits and twelve flaps. The number of slits and flaps can also be provided with less than eight, such as four flaps and two slits with one slit between two adjacent flaps. In other words, with reference to FIG. 9, flaps and slits can be formed only partially around the circumference of the body 82 and not completely around the circumference of the body.

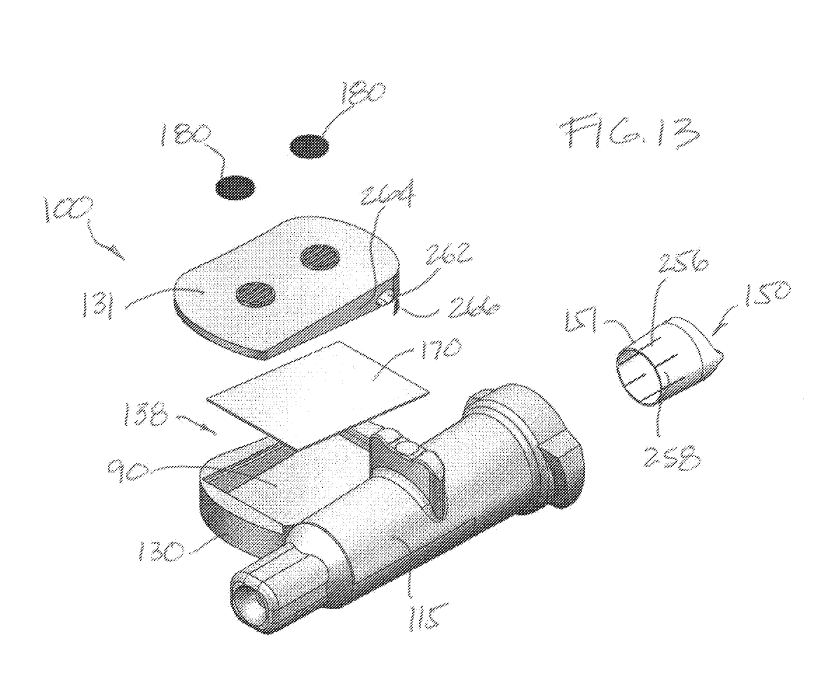

When the barrier 150 of FIG. 9 is placed inside a catheter hub, such as shown with reference to FIG. 12, at least one of the flaps 258 can be positioned over the outlet port or second port 126 of the catheter hub. When the flaps 258 are pushed outwardly by fluid pressure inside the catheter hub 115, the flaps 258 are pushed against the interior surfaces of the catheter hub 115 to limit or restrict flow through the second port 126 from the distal interior cavity 124. Fluid flow from the enclosed compartment 138 through the second port 126 and into the catheter hub 115, such as from fluid infusion into the distal interior cavity 124, is permitted. Fluid pressure can force the one or more flaps 258 to deflect radially inwardly away from the interior wall surfaces towards a central axis or lengthwise axis of the barrier 150 to thereby permit fluid flow from the enclosed compartment 138 into the catheter hub 115 while blocking or limiting fluid flow in the reverse direction, such as preventing blood flow from the distal interior cavity 124 into the enclosed compartment 138 via or through the second port 126.

Thus, the barrier 150 in accordance with aspects of the present alternative embodiment is configured to seal or limit fluid flow in the distal direction through the slit 153, similar to the barrier 150 discussed above with reference to FIG. 5, and also functions as a check valve to limit flow through the second port 240 substantially along only one flow direction. For example, the flaps 258, acting as a check valve, can permit fluid from the enclosed compartment 138 through the second port 126 and into the distal interior cavity 124 but not in the reverse direction. In the reverse direction, fluid pressure presses the flaps 258 against the interior wall surfaces of the catheter hub 115 to seal the second port 126.

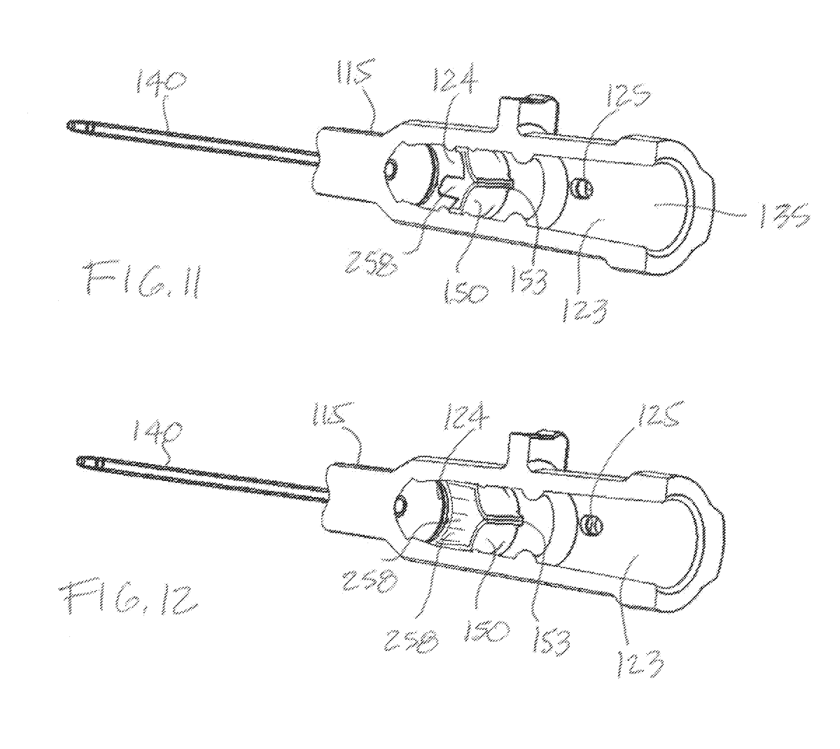

FIG. 10 shows a barrier 150 in accordance to further alternative aspects of the prevent disclosure. The barrier 150 of the present embodiment has an end wall 84 with a tip 152 and a slit 153, similar to the barrier 150 shown and described with reference to FIG. 5. In the present embodiment, the body 82 is provided with an extension 258 that extends from a distal end 252 of the body 82. The extension 258 can serve as or be considered a flap for covering a second port 126 of the catheter hub 115, similar to the flaps 258 discussed above with reference to FIG. 9. However, in the present embodiment, the extension 258 is provided without adjacent flaps and/or without slits.

In some examples, the barrier 150 of FIG. 10 may be provided with two spaced apart extensions 258 for use with a catheter device having two second ports 126 for fluid communication with two enclosed compartments 138, as discussed above. A second extension 258 is partially shown with the barrier 150 of FIG. 10, which is spaced from the extension 258 shown at the top of FIG. 10. Thus, the barrier 150 of FIG. 10 may be used with a catheter device having two second ports 126 for fluid communication with two enclosed compartments 138. The same barrier 150 with two extensions 258 can also be used in a catheter device with a single second port for fluid communication with a single enclosed compartment. In yet other examples, the barrier 150 is provided with only a single extension for use with a catheter hub with a single second port. The one or more extensions or flaps 258 can function like a check valve. For example, the one or more flaps, acting as a check valve, can permit fluid from the enclosed compartment 138 through the second port 126 and into the distal interior cavity 124 but not in the reverse direction. In the reverse direction, fluid pressure presses the one or more flaps 258 against the interior wall surfaces of the catheter hub 115 to seal the second port 126. If used with two second ports 126, two or more flaps 258 can be pressed against the interior wall surfaces of the catheter hub 115 to seal the two second ports.

FIG. 11 shows a cross-sectional side perspective view of a catheter hub 115, similar to that of FIG. 3, but with the barrier 150 of FIG. 10. As shown, the extension 258 on the barrier 150 covers the second port located in the distal interior cavity 124. Thus, any fluid present in the distal interior cavity 124 can be prevented by the extension or flap 258 of the barrier 150 pressed against the interior wall surfaces of the catheter to prevent fluid flow from flowing through the blocked second port 126 and into the enclosed compartment 138 located externally of the catheter hub 115. This can help to prevent clogging or contaminating the filter located in the enclosed compartment by fluid flowing from the distal interior cavity 124 into the enclosed compartment.

FIG. 12 shows a cross-sectional side perspective view of a catheter hub 115, similar to that of FIG. 3, but with the barrier 150 of FIG. 9. As shown, one or more flaps 258 on the barrier 150 can cover the second port located in the distal interior cavity 124. Thus, any fluid present in the distal interior cavity 124 can be prevented by the one or more flaps 258 of the barrier 150 pressing against the interior wall surfaces to prevent fluid flow from flowing through the blocked second port and into the enclosed compartment 138 located externally of the catheter hub. This can help to prevent clogging or contaminating the filter located in the enclosed compartment by fluid flowing from the distal interior cavity 124 into the enclosed compartment.

FIG. 13 is an exploded view of a catheter device 100 with a catheter hub 115, similar to that of FIG. 2 with a few exceptions. In the present embodiment, the catheter tube, needle, needle hub, needle guard, and blood stopper have been omitted for clarity. Further, in the present embodiment, the barrier 150 is shown with an elongated cylindrical base 151 having a plurality of slits 256 and a plurality of flaps 258, similar to the barrier of FIG. 9, and the cover or first component 131 is shown having a port 262 formed through a sidewall 264 of the cover 131. The port 262 on the cover or first component 131 is configured to align with the second port 126 of the catheter hub 115 when the cover 131 is placed over the opening of the first component 130 to enclose the filter 170 therein to assemble the enclosed compartment 138. In other words, the port 262 of the cover and the second port 126 of the catheter hub can be aligned in fluid communication with one another.

In the present embodiment, a flap 266 is provided with the cover 131. The flap 266 is configured to operate as a one-way valve or a check valve to permit fluid flow from the enclosed compartment 138 through the port 262 of the cover and the second port 126 of the catheter hub and into the distal interior cavity of the catheter hub but not permit or at least severely restrict fluid flow from the distal interior cavity of the catheter hub into the enclosed compartment 138. Thus, the flap 266 on the cover 131 can function as a limiting means to limit fluid flow in a similar manner as the flaps on the barrier 150 of FIGS. 9 and 10. The flap 266 on the cover 131 is configured to be pushed against the sidewall 264 of the cover to restrict or limit fluid flow into the enclosed compartment 138. Note that with the flap 266 on the cover 131 of the enclosed compartment 138, the barrier 150 used with the catheter hub can be a simple barrier without any flap, similar to the barrier of FIG. 5.

Methods of making and of using the catheter devices shown and described elsewhere herein are within the scope of the present disclosure.

Although limited embodiments of the catheter devices and their components have been specifically described and illustrated herein, many modifications and variations will be apparent to those skilled in the art. Furthermore, it is understood and contemplated that features specifically discussed for one needle device embodiment may be adopted for inclusion with another needle device embodiment, provided the functions are compatible. For example, release element may be integrated with the needle guard. Accordingly, it is to be understood that the needle devices and their components constructed according to principles of the disclosed device, system, and method may be embodied other than as specifically described herein. The disclosure is also defined in the following claims.

What is claimed is:

1. A catheter device comprising:
a catheter hub comprising a catheter hub body, the catheter hub body having a proximal end, an interior cavity, and a distal end communicating with the proximal end;
a catheter tube attached to the distal end of the catheter hub body;
a barrier located in the interior cavity of the catheter hub body and separating the interior cavity into a distal interior cavity and a proximal interior cavity, the barrier allowing flow in a proximal direction for aspiration but preventing flow in a distal direction through the barrier,
a bypass comprising a first port for directing flow outside the interior cavity and around the barrier and a second port spaced from the first port for direction flow back into the interior cavity; and
a needle projecting through the barrier, through the catheter tube, and having a needle tip extending distally of a distal end of the catheter tube in a ready to use position, the needle having a proximal end attached to a needle hub.

2. The catheter device of claim 1, further comprising a gas permeable membrane communicating with the catheter hub and configured for expelling air outside the catheter hub.

3. The catheter device of claim 1, wherein the first port and the second port are in fluid communication with an enclosed compartment.

4. The catheter device of claim 3, wherein the enclosed compartment extends laterally from the catheter hub body.

5. The catheter device of claim 4, further comprising a filter located in the enclosed compartment, the filter for filtering contaminants passing through the enclosed compartment.

6. The catheter device of claim 1, wherein the first port and the second port are located on different elevations relative to a lengthwise axis of the catheter hub.

7. The catheter device of claim 6, wherein the filter separates the enclosed compartment into a first compartment and a second compartment.

8. The catheter device for claim 7, further comprising at least one gas permeable membrane attached to the enclosed compartment for expelling air.

9. The catheter device of claim 8, wherein the at least one gas permeable membrane is made from PTFE or PDMS.

10. The catheter device of claim 9, further comprising a membrane cover covering the at least one gas permeable membrane to prevent touch contamination of the at least one gas permeable membrane.

11. The catheter device of claim 1, further comprising a needle guard slidably mounted relative to the needle for covering the needle tip.

12. The catheter device of claim 11, wherein the needle guard comprises a proximal wall with an opening and at least one resilient arm.

13. The catheter device of claim 1, wherein the barrier comprises a duckbill valve, a Heimlich valve, or a joker valve.

14. The catheter device of claim 1, further comprising an interior change in profile in the interior cavity of the catheter hub engaging a needle guard.

15. The catheter device of claim 1, wherein the bypass comprises a first component attached to a second component comprising a compartment.

16. The catheter device of claim 1, wherein the second component comprises a bottom wall and a sidewall.

17. The catheter device of claim 1, wherein the barrier comprises a flap for blocking the second port.

18. The catheter device of claim 16, wherein the first component comprises a flap for blocking the second port.

19. A method of manufacturing a catheter device comprising:
attaching a catheter tube to a distal end of a catheter hub, said catheter hub comprising a catheter hub body comprising an interior cavity, a proximal end and a distal end;
placing a barrier in the interior cavity of the catheter hub to define a proximal interior cavity and a distal interior cavity, said barrier is sized and shaped to allow fluid to flow proximally through the barrier from the distal interior cavity to the proximal interior cavity but prevents fluid from flowing distally through the barrier;
projecting a needle through the barrier and through the catheter tube so that a needle tip of the needle extends distally of a distal end of the catheter tube in a ready to use position; said needle having a proximal end attached to a needle hub; and
providing a bypass around the barrier for fluid flow around the barrier.

20. The method of claim 19, further comprising placing a filter in a chamber of the bypass.

21. The method of claim 19, further comprising placing a needle guard slidably relative to the needle for covering the needle tip in a protective position.

22. The method of claim 20, wherein the bypass comprises a first port in communication with the proximal interior cavity, a second port in communication with the distal interior cavity, and an enclosed compartment in communication with both the first port and the second port.

23. A method of using a catheter device comprising a catheter hub with a catheter tube, a needle attached to a needle hub, and a barrier located in an interior cavity of the catheter hub, said method comprising:
removing the needle and needle hub from the catheter hub, the barrier, and the catheter tube;
placing a male medical implement into a proximal opening of the catheter hub; and
passing fluid from the male medical implement through a filter prior to discharging the fluid out through the catheter tube; and
wherein the barrier prevents fluid from flowing distally through the barrier but allows fluid to flow proximally through the barrier.
